(19) United States
(12) Patent Application Publication
(10) Pub. No.: US 2024/0173063 A1
(43) Pub. Date: May 30, 2024

(54) SYSTEM AND METHOD FOR ABLATING A TISSUE SITE BY ELECTROPORATION WITH REAL-TIME MONITORING OF TREATMENT PROGRESS

(71) Applicants: Virginia Tech Intellectual Properties Inc., Blacksburg, VA (US); AngioDynamics, Inc., Latham, NY (US)

(72) Inventors: Robert Neal, II, Richmond, VA (US); Rafael V. Davalos, Aptos, CA (US)

(21) Appl. No.: 18/528,051

(22) Filed: Dec. 4, 2023

Related U.S. Application Data
(63) Continuation of application No. 17/338,960, filed on Jun. 4, 2021, now Pat. No. 11,890,046.
Application No. 17/338,960 is a continuation of application No. 16/280,511, filed on Feb. 20, 2019, now abandoned.
Application No. 16/280,511 is a continuation of application No. 14/940,863, filed on Nov. 13, 2015, now Pat. No. 10,238,447.
Application No. 14/940,863 is a continuation-in-part of application No. 14/012,832, filed on Aug. 28, 2013, now Pat. No. 9,283,051.
Application No. 14/012,832 is a continuation-in-part of application No. 12/491,151, filed on Jun. 24, 2009, now Pat. No. 8,992,517.
Application No. 12/491,151 is a continuation-in-part of application No. 12/432,295, filed on Apr. 29, 2009, now Pat. No. 9,598,691.
(60) Provisional application No. 62/079,061, filed on Nov. 13, 2014.
(60) Provisional application No. 62/173,538, filed on Jun. 10, 2015.
(60) Provisional application No. 61/171,564, filed on Apr. 22, 2009.
(60) Provisional application No. 61/167,997, filed on Apr. 9, 2009.
(60) Provisional application No. 61/075,216, filed on Jun. 24, 2008.
(60) Provisional application No. 61/125,840, filed on Apr. 29, 2008.

(51) Int. Cl. A61B 18/12; A61B 18/00; A61B 18/14; A61B 18/18; A61B 34/10; C12N 13/00

(52) U.S. Cl. CPC A61B 18/1206; A61B 18/14; A61B 18/1477; A61B 18/18; A61B 34/10; C12N 13/00; A61B 2018/0016; A61B 2018/00577; A61B 2018/00613; A61B 2018/00666; A61B 2018/00672; A61B 2018/00678; A61B 2018/00767; A61B 2018/00875; A61B 2018/1425; A61B 2034/104

(57) ABSTRACT

A medical system for ablating a tissue site with real-time monitoring during an electroporation treatment procedure. A pulse generator generates a pre-treatment (PT) test signal prior to the treatment procedure and intra-treatment (IT) test signals during the treatment procedure. A treatment control module determines impedance values from the PT test signal and IT test signals and determines a progress of electroporation and an end point of treatment in real-time based on the determined impedance values while the treatment progresses.

### Cross Reference to Related Applications

The present application claims priority to and is a Continuation application of U.S. application Ser. No. 16/280,511, filed on Feb. 20, 2019, which published as U.S. Patent Application Publication No. 2019/0175248 on Jun. 13, 2019, which is a Continuation application of U.S. application Ser. No. 14/940,863, filed on Nov. 13, 2015, which published as U.S. Patent Application Publication No. 2016/0066977 on Mar. 10, 2016, and which issued as U.S. Pat. No. 10,238,447 on Mar. 26, 2019. The '863 application claims priority to and the benefit of the filing date of U.S. Provisional Application No. 62/079,061 filed on Nov. 13, 2014, and U.S. Provisional Application No. 62/173,538 filed on Jun. 10, 2015. The '863 application is also a Continuation-in-Part (CIP) application of parent application U.S. application Ser. No. 14/012,832, filed on Aug. 28, 2013, which published as U.S. Patent Application Publication No. 2013/0345697 on Dec. 26, 2013 and issued as U.S. Pat. No. 9,283,051 on Mar. 15, 2016. The '832 application is a Continuation-in-Part (CIP) application of U.S. application Ser. No. 12/491, 151, filed on Jun. 24, 2009, which published as U.S. Patent Application Publication No. 2010/0030211 on Feb. 4, 2010, and issued as U.S. Pat. No. 8,992,517 on Mar. 31, 2015. The '151 application claims priority to and the benefit of the filing dates of U.S. Provisional Patent Application Nos. 61/171,564, filed on Apr. 22, 2009, 61/167,997, filed on Apr. 9, 2009, and 61/075,216, filed on Jun. 24, 2008, and the '151 application is a Continuation-in-Part application of U.S. patent application Ser. No. 12/432,295, filed on Apr. 29, 2009, which published as U.S. Patent Application Publication No. 2009/0269317 on Oct. 29, 2009 and issued as U.S. Pat. No. 9,598,691 on Mar. 21, 2017. The '295 application claims priority to and the benefit of the filing date of U.S. Provisional Patent Application No. 61/125,840, filed on Apr. 29, 2008. All of these applications, publications and patents are incorporated by reference herein in their entireties.

### FIELD OF THE INVENTION

The present invention relates to a control system for controlling an electroporation medical treatment device and more particularly, to such devices with real- time monitoring of electroporation treatment progress.

### BACKGROUND OF THE INVENTION

Medical devices for delivering therapeutic energy such as electrical pulses to tissue include one or more electrodes and a pulse generator. The pulse generator allows the electrode to deliver the therapeutic energy to a targeted tissue, thereby causing ablation of the tissue.

Electroporation procedure parameters that influence the size and shape of their affected region include the nature of the tissue (cellularity, extracellular constituent composition, anisotropy, conductivity, metabolic demand), patient specific anatomy, the pulse delivery apparatus (number of electrodes, their size, and relative geometry), and pulse parameters (voltage, number of pulses, pulse length, pulse delivery rate). In addition to the above, the generator's maximum pulse intensity capabilities (maximum voltage and current) dictate the maximum achievable treatment region. Where controllable and large lesions are desired, it is important to maintain pulses that are capable of inducing electroporation effects to the tissue while remaining below the maximum generator capacity.

In conventional electroporation devices, before the treatment procedure a physician would decide on a particular pulse delivery apparatus and select the pulse parameters. As can be appreciated, the electroporation therapy treatment plans selected by the physician are limited to using a retrospective dimension data approach, where a pre-determined pulse parameter protocol is delivered between each electrode pair in an array and the pulse parameters are selected from previously existing ablation data. Once the treatment procedure starts, the electroporation device follows the pre-treatment programming set by the physician and delivers the pulses according to the pre-selected pulse parameters.

However, this approach ignores the specifics of the actual case, which will vary both in terms of initial tissue properties and tissue response to the electroporation pulses for each patient. Specifically, there is no way to monitor the progress of the treatment procedure or alter the settings other than to stop the procedure manually. Thus, even when the procedure completes normally, there was no assurance that there were clinically sufficient electroporation of the targeted region due to the unpredictable nature of patient environments and living tissue.

Therefore, it would be desirable to provide a system and method for monitoring the progress of an electroporation treatment procedure in real-time and to determine in real-time whether an end point has been reached for particular patients.

### SUMMARY OF THE DISCLOSURE

According to one aspect of the present invention, a medical system for ablating a tissue site with real-time monitoring during an electroporation treatment procedure includes at least two electrodes and a pulse generator configured to generate electroporation pulses for ablation of tissue in a target region. The pulse generator also generates a pre-treatment (PT) test signal having a frequency of at least 1 MHz prior to the treatment procedure and intra-treatment (IT) test signals during the treatment procedure. Use of the PT test signal provides a baseline value that is specific to the patient being treated. A treatment control module determines impedance values from the PT test signal and IT test signals and determines a progress of electroporation in real- time based on the determined impedance values while the treatment procedure progresses.

According to another aspect of the present invention, a method of determining a progress of an electroporation treatment procedure for ablating a tissue site is provided. The method applies a PT test signal having a frequency of at least 1 MHZ to a target region of a tissue site through at least one electrode and determines an impedance value based on the applied PT test signal. Use of the PT test signal provides a baseline impedance value that is specific to the patient being treated. During the treatment procedure, a plurality of IT test signals are applied and an impedance value for each applied IT test signal is determined. The method then determines a progress of electroporation of the target tissue site, based on the determined impedance values of the IT test signals and PT test signal. The method can also determine an end of treatment based on the determined impedance values of the IT test signals and PT test signal.

Advantageously, use of a baseline value which is specific to the particular patient being treated for monitoring the progress and determining an end of treatment in real-time will result in improved treatment delivery and improved likelihood for successful outcome.

### BRIEF DESCRIPTION OF THE DRAWINGS

FIG. 1 is a block diagram of an electroporation device according to one aspect of the present invention.

FIG. 2 is a block diagram of a treatment control computer of FIG. 1.

FIG. 3 is a block diagram of a pulse generator shown in FIG. 1.

FIG. 4 is a block diagram of a sensor of FIG. 3.

FIG. 5 is a flowchart of a method of ablating a tissue site by electroporation with real-time monitoring of treatment progress during an electroporation procedure.

FIG. 6 is a schematic depiction of successful and failed electroporation treatment procedures.

FIG. 7 is a screen shot of an electroporation treatment procedure in progress with real-time monitoring of the treatment progress.

FIG. 8 is a graph of predicted impedance values across a frequency spectrum as predicted by a Cole model and a superimposed impedance values from a rat liver.

### DETAILED DESCRIPTION OF THE INVENTION

Throughout the present teachings, any and all of the one, two, or more features and/or components disclosed or suggested herein, explicitly or implicitly, may be practiced and/or implemented in any combinations of two, three, or more thereof, whenever and wherever appropriate as understood by one of ordinary skill in the art. The various features and/or components disclosed herein are all illustrative for the underlying concepts, and thus are non-limiting to their actual descriptions. Any means for achieving substantially the same functions are considered as foreseeable alternatives and equivalents, and are thus fully described in writing and fully enabled. The various examples, illustrations, and embodiments described herein are by no means, in any degree or extent, limiting the broadest scopes of the claimed inventions presented herein or in any future applications claiming priority to the instant application.

In the present specification, the voltage value of any AC signals refers to root mean square (RMS) voltage, rather than peak voltage, unless specifically mentioned otherwise. Throughout the present specification, tissue properties are discussed in terms of an impedance. Both impedance and conductance measure the resistance of tissue in passing current. Thus, any reference to an impedance of tissue necessarily encompasses conductivity and vice versa.

In the present invention, biofeedback in the form of local bulk tissue electrical properties and their change in response to electroporation (EP) or irreversible electroporation (IRE) therapy are used to guide the user and indicate the extent or progress of electroporation during the electroporation procedure and to determine an end point for the procedure while the procedure is in progress. Specifically, impedance of test signals is used to enable a refined determination of EP effects and dimension, thus improving tolerances for ablation dimensions and ensuring successful electroporation in the desired regions. Ultimately, this will result in improved treatment delivery, more accurate determination of treated tissue dimensions and margins, provide users with feedback information on determining success of the procedure, faster treatment times, and improved likelihood for successful ablation outcomes in EP ablation treatment.

The tissue properties can be measured through dedicated measurement electrodes, or through the same electrodes that apply electroporation pulses. In the case of the latter, if there are more than one pair of electrodes that apply electroporation pulses, e.g., 2 pairs, the tissue property measurements can be made with one pair while the other pair is applying electroporation treatment pulses. The measurements are generally made during the quiet times between electroporation pulses.

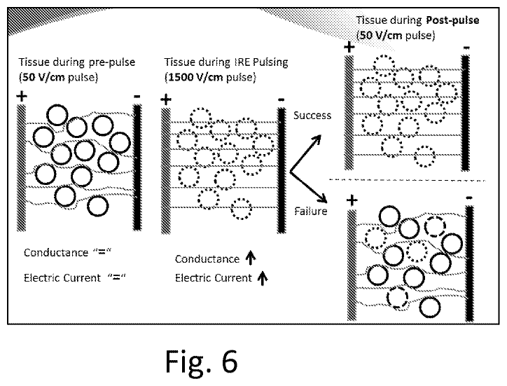

It relies on the fact that electroporated cell membranes no longer serve a significant barrier to electrolyte mobility and electrical current flow through the cells in tissue. In turn, electroporated regions will increase the bulk tissue conductivity. When irreversible, this change at the cellular level is permanent. This increased conductivity may then serve as an artifact to indicate the extent of tissue electroporation. As a result, it is likely that pulse metric behavior and trends correlate with ablation dimensions. By measuring and analyzing tissue property changes, such as a change in conductivity and/or impedance, that result from the electroporation therapy, the present invention deduces the dimensions or completeness of an IRE lesion and uses this information to control the progress of an electroporation therapy. This information provides the user with feedback that can be used to set or adjust the pulse parameters (voltage, pulse length, number of pulses) to tailor the treatment protocol to the specific patient, and/or indicate when a particular electrode pair has satisfied their required ablation dimensions to attain complete coverage of the targeted region. This phenomenon is illustrated in FIG. 6. Prior to the treatment procedure, as shown in the left image, electrical current from a DC test voltage of 50 V/cm flows around the cells without electroporation because the DC current cannot penetrate the cell membranes (shown as dark circles). The impedance/conductance and current are steady and do not change from one test pulse to the next. Once electroporation pulses start to be delivered as the middle image shows, however, the membranes start to develop holes and electrical current start to flow through the punctured cell membranes (shown as dotted circles). As a result, conductance and current from the DC test pulse increase from one test pulse to the next.

If the irreversible electroporation treatment procedure was successful, the electroporated cells would be unable to close the membrane holes, and the electrical current and the associated impedance/conductance from the test pulses would stabilize to certain predetermined known values. However, if the irreversible electroporation treatment procedure was not successful, that means either at least some of the cells were able to repair and close the holes or membranes of some of the cells were never punctured. In that case, the current would only be able to flow through the punctured membranes and not through the repaired cell membranes. As a result, the current and conductance would not reach the known threshold values.

There are several possible methods of determining the progress and end point of a treatment procedure.

One method uses a desired ultimate current value for a given electrode geometry and pulse protocol to indicate completeness or progress (e.g., percentage of completion) of the treatment procedure. Preferably, the current values are obtained from test signals prior to and in between IRE treatment pulses. If the desired threshold value is not reached, the voltage or pulse length could be increased, or the application of treatment electroporation pulses would continue until the threshold value is reached by relying on the concept of a current-creep where electrical currents have a tendency to increase over the course of a procedure due to increasing electroporated volume and cell electroporation density as well as cumulative temperature increases. An exemplary threshold current value may be 0.35 Amps.

Another method is to use a relative/changing current. Rather than relying on a threshold target current, this procedure relies on a change in the electrical current in the test signals to indicate the changing tissue properties over the course of an IRE sequence between electrodes. Preferably, this method would employ an averaging algorithm, such as a simple moving average (SMA) or exponential moving average (EMA) over several consecutive test signal, which provides for a more stable evaluation of electric current change, and factors out signal-by-signal anomalies in current. As an example, two SMA's would be used with one lagging the other by several test signals.

It uses either a difference in current relative to that from an earlier pulse (Δi=iₖ−i₀) or as a relative change (% Δi=Δi/i₀) as the threshold value. As a very simple example, the threshold difference in current can be 0.02 Amps and the threshold relative change in percentage can be 1.0%. If the desired threshold value is not reached, pulsing would continue until it does (relying on current-creep while factoring out thermal influence on rise in current) or the voltage or pulse length could be increased based on user input.

Another method is to use a desired final threshold impedance value (either absolute impedance value or relative/changing impedance value) for a given electrode geometry and pulse protocol to indicate completeness or progress (e.g., percentage of completion) of the treatment procedure. Similar to the method discussed above with respect to measuring the current values, this method would also employ an averaging algorithm, such as a simple moving average (SMA) or exponential moving average (EMA) over several consecutive test signal, which provides for a more stable evaluation values and factors out signal-by-signal anomalies.

In a preferred embodiment, only the real part of the impedance value is used. If using the absolute impedance value (preferably the real part of impedance if AC test signals are used), pulsing is continued until the desired threshold value (e.g., 150 Ohm) is reached. The desired threshold value can be derived from a pre-treatment test signal and/or the type of tissue being treated. If the desired threshold impedance value is not reached, the voltage or pulse length could be increased, or the pulsing would continue until the threshold value is reached by relying on the concept of an impedance-creep where electrical impedance has a tendency to decrease over the course of a procedure due to increasing electroporated volume and cell electroporation density, while controlling to factor out current-creep due to cumulative temperature increases.

If using a relative impedance value (preferably the real part of impedance) as the threshold value, a change in the electrical impedance can be monitored for indicating the changing tissue properties over the course of an IRE sequence between electrodes. It uses either a difference in impedance relative to that from an earlier pulse (ΔR=Rₖ−R₀) or as a relative change (% ΔR=ΔR/R₀) as the threshold value. For example, the threshold difference in impedance can be 10 Ohms and the threshold relative change in percentage can be 1.0%. If the desired threshold value is not reached, pulsing would continue until it does (relying on impedance-creep) or the voltage or pulse length could be increased based on user input. An advantage of using impedance values is that the influence of what voltage is applied is factored out, resulting in a more accurate and reliable method of monitoring the treatment progress and determining the end point of the treatment procedure.

Using pulse metrics derived from the actual therapeutic EP or IRE pulses to indicate tissue properties and their response to electroporation pulses does not account for the transiently altered tissue properties resulting from reversibly electroporated cells. As a result, monitoring purely therapy pulse metrics may give a false-read on the extent of electroporation, due to the 2 to 5-fold increase in electrical conductivity of many tissues during electroporation pulses. To more effectively attain a determination of the completeness and size of irreversibly electroporated tissue, a low-strength test signal can be applied between adjacent therapy pulses or sets of pulses. This low-voltage test signal would not inherently permeabilize the cells, and thus changes in its current or resistance may better indicate bulk tissue property changes resulting from IRE. Thus, in utilizing this type of indicator to control pulse protocols, the changes indicated would then mimic those aforementioned for the therapy pulses (current and resistance; absolute thresholds and relative changes). An upper limit for the desired post-pulse resistance could potentially be derived from the effective resistance of the tissue during the electroporation therapy pulses.

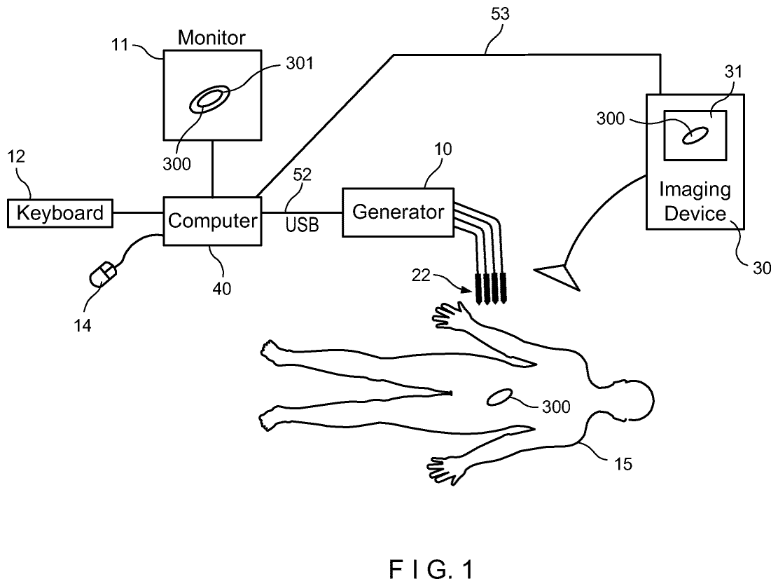

One embodiment of the present invention is illustrated in FIG. 1. The components used with the present invention are illustrated in FIG. 1. One or more electrodes/probes 22 deliver therapeutic energy and are powered by a voltage pulse generator 10 that generates high voltage pulses as therapeutic energy such as pulses capable of irreversibly electroporating the tissue cells. In the embodiment shown, the voltage pulse generator 10 includes six separate receptacles for receiving up to six individual probes 22 which are adapted to be plugged into the respective receptacle. The receptacles are each labeled with a number in consecutive order. In other embodiments, the voltage pulse generator 10 can have any number of receptacles for receiving more or less than six probes.

Each probe 22 includes either a monopolar electrode, bipolar electrodes having at least two electrodes (electrode conducting regions) separated by an insulating sleeve, or multipolar electrodes having greater than two electrode surfaces separated by one or more insulating sleeves which can be energized simultaneously or at different times. In one embodiment, if the probe includes a monopolar electrode, the amount of exposure of the active portion of the electrode can be adjusted by retracting or advancing an insulating sleeve relative to the electrode. See, for example, U.S. Pat. No. 7,344,533, which is incorporated by reference herein. In the embodiment shown, the probes 22 are monopolar electrodes. The generator 10 is connected to a treatment control computer 40 having input devices such as keyboard 12 and a pointing device 14, and an output device such as a display device 11 for viewing an image of a target treatment area such as a lesion 300 surrounded by a safety margin 301. The therapeutic energy delivery device 20 is used to treat a lesion 300 inside a patient 15. An imaging device 30 includes a monitor 31 for viewing the lesion 300 inside the patient 15 in real time. Examples of imaging devices 30 include ultrasonic, CT, MRI and fluoroscopic devices as are known in the art.

For purposes of this application, the terms “code”, “software”, “program”, “application”, “software code”, “software module”, “module” and “software program” are used interchangeably to mean software instructions that are executable by a processor.

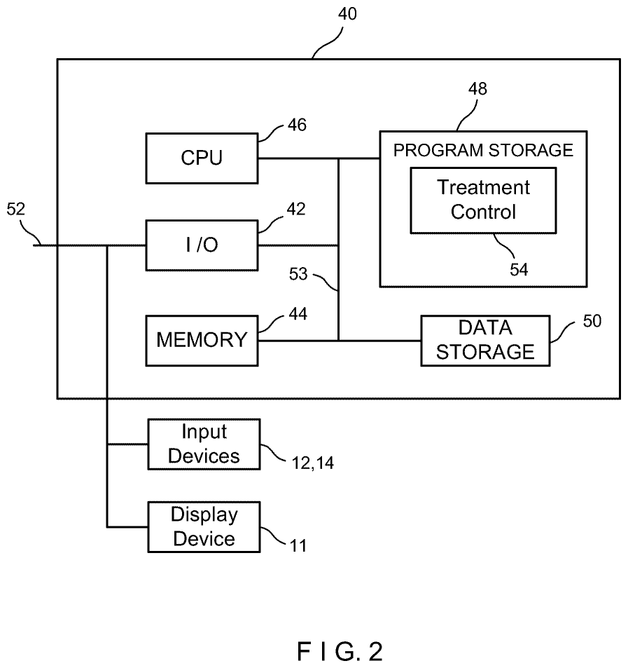

The “user” can be a physician or other medical professional. The treatment control module 54 (FIG. 2) executed by a processor outputs various data including text and graphical data to the monitor 11 associated with the generator 10.

Referring now to FIG. 2, the treatment control computer 40 of the present invention is connected to the communication link 52 through an I/O interface 42 such as a USB (universal serial bus) interface, which receives information from and sends information over the communication link 52 to the voltage generator 10. The computer 40 includes memory storage 44 such as RAM, processor (CPU) 46, program storage 48 such as ROM or EEPROM, and data storage 50 such as a hard disk, all commonly connected to each other through a bus 53. The program storage 48 stores, among others, computer software (treatment control module 54) which assists a user/physician to plan for, execute, and review the results of a medical treatment procedure. The treatment control module 54, executed by the processor 46, assists a user to plan for a medical treatment procedure by enabling a user to more accurately position each of the probes 22 of the therapeutic energy delivery device 20 in relation to the lesion 300 in a way that will generate the most effective treatment zone. The treatment control module 54 can display the anticipated treatment zone based on the position of the probes and the treatment parameters. Using any of the above described methods, the treatment control module 54 can display the progress of the treatment in real time and can display the results of the treatment procedure after it is completed. This information can be used to determine whether the treatment was successful and whether it is necessary to re-treat the patient.

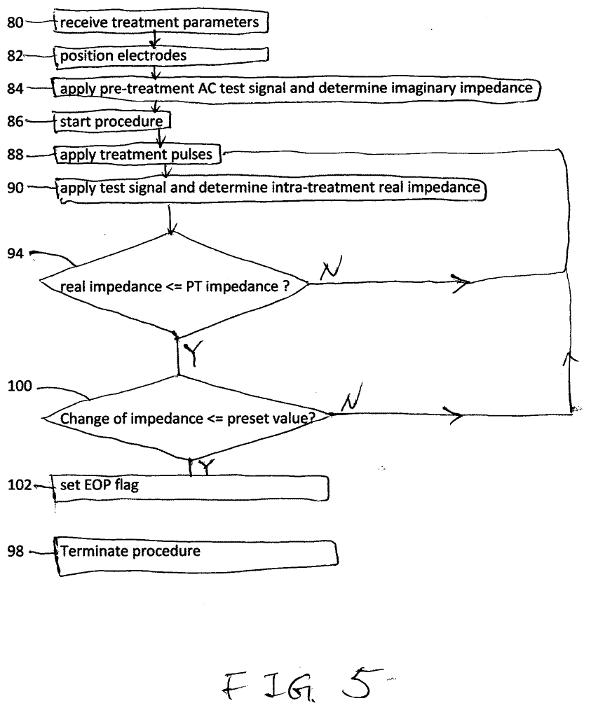

The module 54 is also adapted to monitor and display the progress of the electroporation procedure and to determine a successful end point based on the electrical properties of the tissue prior to and during the treatment procedure as will be explained in more detail with reference to FIG. 5. Being able to in real-time monitor and see the end point of the treatment procedure is a huge advantage over the current method in which the physician is performing the treatment essentially blindly without having any idea about whether the treatment is progressing or at what point the treatment procedure is finished.

The program storage 48 stores various electrical threshold values that are used to monitor the treatment procedure. When the programmed sequence of pulses have been delivered and the end point of the procedure has not been reached, the user interface portion of the control module 54 retrieves the recommended parameter changes from the database and presents them to the user through the display 11. The treatment control module 54 can also change the threshold values for determining the progress and the end point of the procedure based on initial treatment pulse parameters programmed by the user. For example, different body parts/organs or different health/age of patients may require different thresholds as their conductivity and susceptibility to irreversible electroporation may differ. User can manually input the various thresholds for different tissue types or the system can have these thresholds stored electronically.

Alternatively, the treatment control module 54 can also automatically derive or adjust the threshold values for determining the progress and the end point of the procedure based on test signals (e.g., AC test signals) that are applied and determining electrical properties of the cells such as impedance values. The control module 54 may then store the changed threshold values in the program storage 48 for later use as the new criteria for comparison.

Further, AC intra-treatment test signals may continue to be delivered in addition to the comparative DC intra-treatment test signals. By tracking the change in impedance for the AC-signal, the treatment control module 54 determines and factors out the effects on impedance occurring due to temperature rise. This enables more accurately tracking changes in the real-part of the impedance by reflecting changes encountered solely due to persistent electroporated cells. A more detailed discussion of the control module 54 will be made later herein with reference to FIG. 5.

Any of the software program modules in the program storage 48 and data from the data storage 50 can be transferred to the memory 44 as needed and is executed by the CPU 46.

In one embodiment, the computer 40 is built into the voltage generator 10. In another embodiment, the computer 40 is a separate unit which is connected to the voltage generator through the communications link 52. The communication link 52 can be, for example, a USB link.

In one embodiment, the imaging device 30 is a stand-alone device which is not connected to the computer 40. In the embodiment as shown in FIG. 1, the computer 40 is connected to the imaging device 30 through a communications link 53. As shown, the communication link 53 is a USB link. In this embodiment, the computer can determine the size and orientation of the lesion 300 by analyzing the data such as the image data received from the imaging device 30, and the computer 40 can display this information on the monitor 11. In this embodiment, the lesion image generated by the imaging device 30 can be directly displayed on the monitor 11 of the computer running the treatment control module 54. This embodiment would provide an accurate representation of the lesion image on the grid 200, and may eliminate the step of manually inputting the dimensions of the lesion in order to create the lesion image on the grid 200. This embodiment would also be useful to provide an accurate representation of the lesion image if the lesion has an irregular shape.

It should be noted that the software can be used independently of the generator 10. For example, the user can plan the treatment in a different computer as will be explained below and then save the treatment parameters to an external memory device, such as a USB flash drive (not shown). The data from the memory device relating to the treatment parameters can then be downloaded into the computer 40 to be used with the generator 10 for treatment.

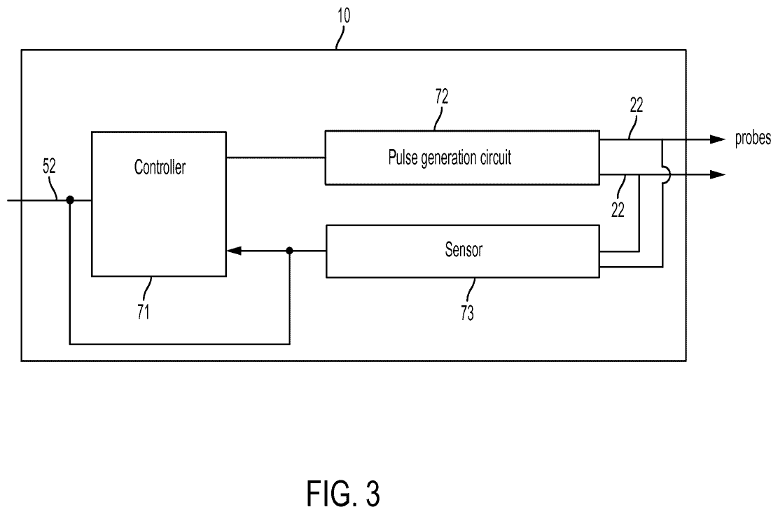

FIG. 3 is a functional block diagram of a pulse generator 10 shown in FIG. 1. FIG. 2 illustrates one embodiment of a circuitry to monitor the progress of and determine an end point of the treatment procedure. A USB connection 52 carries instructions from the user computer 40 to a controller 71. The controller 71 can be a computer similar to the computer 40 as shown in FIG. 2. The controller 71 can include a processor, ASIC (application-specific integrated circuit), microcontroller or wired logic. The controller 71 then sends the instructions to a pulse generation circuit 72. The pulse generation circuit 72 generates the pulses and sends electrical energy to the probes. For clarity, only one pair of probes/electrodes is shown. However, the generator 10 can accommodate any number of probes/electrodes such as 6 probes. In the embodiment shown, the pulses are applied one pair of electrodes at a time, and then switched to another pair. The pulse generation circuit 72 includes a switch, preferably an electronic switch that switches the probe pairs based on the instructions received from controller 71.

A sensor 73 can sense the current and voltage between each pair of the probes in real time and communicate such information to the controller 71, which in turn, communicates the information to the computer 40. Although the treatment control module 54 houses the software code for monitoring the treatment procedure, it may be beneficial for the controller 71 to store such module as the speed of monitoring can be important in some cases.

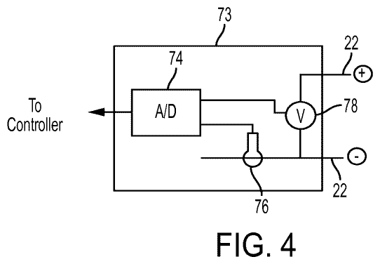

FIG. 4 is a functional block diagram of a sensor 73 of FIG. 3. The sensor 73 includes a voltage sensor 78 connected across a pair of electrodes 22 and a current sensor 78 connected to a negative electrode (return conduit) in the pair of electrodes. Although FIGS. 3-4 show two electrodes from two wires 22, there may be multiple electrodes between the two wires 22. The sensed values are continuously received and digitized by an A/D converter 74 and transmitted to the controller 71. Preferably, the A/D converter 74 can sample the sensed values at a very fast rate and preferably at a rate of at least 100 MHz (100 million samples per second) for the control module 54 to be able to accurately assess the complex impedance of test signals which may be an AC signal at a relatively high frequency.

The current sensor 76 can be a Hall effect sensor/probe which is positioned around an electrode so as to measure the electric current without directly interfering with the pulse signal. Typically, the current sensor 76 is placed on the negative signal connection of the electrode pair. If the electrode pairs are switched, then only one current sensor connected at the input side of the switch is needed. Otherwise, if there are 3 pairs of electrodes, for example, and all are firing at the same time, there will be 3 current sensors so as to measure the electric current of each pair separately. In that case, the current from the three sensors will need to be added,

The voltage sensor 78 can be a conventional voltage divider, comprised of two serially connected resistors, that measures a voltage drop across a known resistance value. The voltage sensor 78 uses resistors which are of much higher resistance than the tissue (kΩ-MΩ, versus tissue, which is hundreds of Ω), and thus induces negligible effect on the strength of the pulses delivered to the tissue. A correction factor is calculated for the divider circuit based on the resistances of the two resistors in the voltage divider circuit and the resistance of the load (tissue resistance) to determine the true delivered voltage to the tissue based on the measured voltage drop across the resistor.

A method of ablating a tissue site with real-time monitoring during an electroporation treatment procedure will now be explained with reference to FIG. 5.

The steps executed are part of the treatment control module 54 which can be part of the computer 40 or a part of the controller 71 in the pulse generator 10 itself for faster response. Referring to FIG. 5, in step 80, the treatment control module 54 graphically interacts with the user to receive treatment parameters which include voltage between electrodes, electrode separation distance, firing sequence among the electrode pairs, pulse delivery/firing rate, pulse duty cycle, number of pulses in a pulse set/train, number of pulse sets/trains, inter-pulse delay, inter pulse train delay, electrode exposure length, pulse parameter changes for each abnormal condition and the like. In step 82, a user/physician positions the electrodes 22 at a tissue site such that the electroporation field covers the target region. The target region is now ready to be treated.

Prior to the start of the actual treatment procedure (prior to delivering electroporation pulses), however, at least one pre-treatment (PT) test signal is delivered to establish a baseline parameter to be compared to similar parameters during the treatment so that a progress and an end of treatment can be determined during the treatment procedure.

In step 84, the pulse control module 54 instructs the controller 71 in the pulse generator 10 to generate the PT test signal through the electrodes 22 that have been placed in the patient. Preferably, the pulse generation circuit 72 generates an alternating current (AC) sine wave signal as the PT test signal whose voltage amplitude (RMS) is insufficient to cause an electroporation of a majority of tissue cells in the target region, and more preferably is insufficient to cause electroporation of any tissue cells in the target region. Although the voltage of the PT test signal depends to a certain extent on the type of tissue cells to be ablated, it is generally less than 500 volts/cm and is preferably between 10 and 200 volts/cm. The frequency of the PT test signal is between 1 KHz and 2 GHz. More preferably, the frequency of the PT test signal is at least 1 MHz and at most 1 GHz. At 1 MHz, the effect of the cellular membrane on impedance starts to diminish as the current at that frequency would start to bypass the capacitive nature of the membranes.

Most preferably, however, the frequency of the PT test signal is at least 100 MHz at which frequency the cell membrane's effects on impedance is substantially diminished and/or at most 500 MHz. At 100 MHz, the frequency is higher than the Beta dispersion frequency (approximately 1 KHz to 1 MHZ) to minimize the effect of the cellular membranes to resist current flow and maximize the effects of the intra-cellular structures on current flow. The duration of the PT test signal can vary but should be sufficiently long (e.g., 1-10 milliseconds) to establish a stable impedance value. If needed, several PT test signals can be made to ensure that the impedance value is consistent.

While the PT test signal is being applied, the sensor 73 continuously senses the current values which are sent to the controller 71. The treatment control module 54 then determines a complex impedance from the measured current values along with the applied voltage as measured from the voltage sensor 78. In some embodiment, the complex impedance could be in the form of conductance. As well known in the art, the complex impedance can be written as the following equation.

Z=R+JX (1)

R represent the real part, X represents the imaginary part and J represents a phase of the voltage relative to the current. The unit for both R and X is Ω (Ohm). The value of the imaginary impedance X is a positive number in absolute value regardless of whether the voltage leads (+J) or lags (−J) the current. Alternatively, a plurality of PT test signals can be delivered to the electrodes and the complex impedance values are averaged.

Once the complex impedance value is determined, it is stored in the memory 44 by the treatment control module 54 to be used as a baseline to compare against later determined impedance values in order to determine an end of treatment. In one particularly preferred embodiment, the imaginary part X of the complex impedance Z is used as the baseline value because it represents the internal resistance of the cells which excludes the effects on the current flow of the cell membranes.

In some embodiments, the calculated imaginary impedance value may be adjusted down by a selected resistance value (or preselected percentage such as 5-10%) before being stored in the memory 44 as the baseline value in order to account for the fact that the resistance of the tissue cells may decrease by the selected resistance as the applied electroporation pulses increase the temperature of the tissue being ablated. For example, if the obtained impedance value is 150 Ohms (imaginary part of the impedance), a set value of 20 Ohms may be subtracted to account for the fact that the temperature rise may reduce the impedance by that amount. Thus, the value of 130 Ohms may be stored as the baseline value for comparison.

In step 86, based on the received parameters, the treatment control module 54 instructs the controller 71 in the pulse generator 10 to start an electroporation procedure. In step 88, under the control of the controller 71, the pulse generation circuit 72 starts delivering electroporation pulses through the electrodes 22 that have been placed in the patient.

In step 90, while the treatment procedure is in progress, the pulse control module 54 instructs the controller 71 in the pulse generator 10 to generate and apply an intra-treatment (IT) test signal through the electrodes 22. In one embodiment, the IT test signal is generated between electroporation pulses so as not to interfere with the treatment pulses and to receive a cleaner signal. The IT test signal is typically a direct current (DC) signal because only the real part of the impedance value is needed. Alternatively, the IT test signal can be the same type of signal as the PT test signal. In that case, the real part R is used as the comparison against the stored baseline value as will be explained in more detail below.

Preferably, the IT test signal has a voltage whose amplitude is insufficient to cause an electroporation of a majority of tissue cells in the target region, and more preferably is insufficient to cause electroporation of any tissue cells in the target region. Although the voltage of the IT test signal depends to a certain extent on the type of tissue cells to be ablated, it is generally less than 500 volts/cm and is preferably between 10 and 200 volts/cm. Similar to the PT test signal, the duration of the IT test signal can vary but should be sufficiently long (e.g., 10 to 100 microseconds for a DC test signal, 1-10 milliseconds for an AC test signal) to establish a stable impedance value. If needed, several IT test signals can be made to ensure that the impedance value is consistent.

The frequency of applying the IT test signals to obtain comparison values to compare against the baseline value can vary depending on the tissue type being treated and other treatment parameters. Typically, the IT test signal can be applied after every treatment electroporation pulse or after several electroporation pulses. In an alternative embodiment, the IT test signal can be applied after every train of pulses (e.g., after a train of 10 electroporation pulses).

While the IT test signal is being applied, the sensor 73 continuously senses the current values which are sent to the controller 71. The treatment control module 54 then determines an impedance from the measured current values along with the applied voltage. In a preferred embodiment, regardless of whether the IT test signal is an AC or DC signal, the treatment control module 54 determines the real part of the impedance in a known manner and stores it in the memory 44 as a comparison value for comparison against the stored baseline value.

In a preferred embodiment, a progress of the electroporation procedure is determined and displayed on the monitor 11 as the treatment procedure progresses. To do so, prior to the treatment procedure, a second PT test signal (e.g., DC test signal having 50 volts/cm) is applied and a DC resistance value (i.e., real resistance) is obtained. The difference between the baseline value and the DC resistance value from the second PT test signal is obtained and stored in the memory 44. Then, the progress of the treatment can be calculated by dividing a numerator value (comparison resistance value from step 90 less the baseline resistance value) by the difference value to obtain the percentage of ablation that still needs to be completed. As an example, assume that the baseline value and the DC resistance value are 150 Ohms and 600 Ohms, respectively. The difference value then is 450 Ohms. As IT test signals are applied, assume that the comparison resistance values are calculated to be 550, 300, 200 and 170. Then, the progress percentage are calculated as (550-150)/450, (300-150)/450, (200-150)/450, and (170-150)/450. Accordingly, the percentage of ablation that needs to be completed are displayed on the monitor 11 as 89%, 33%, 11% and 4%, respectively. When the number goes to 0%, then the treatment control module 54 determines that an end point of the treatment procedure has been reached. If percentage of completion is desired, of course, the numbers would be subtracted from 100%.

Similarly, the progress of the treatment can be determined from the current values. As an example, assume that the current value from the DC PT test signal is 50 V/600 Ohms=0.08 Amps and the target current value is 50 V/150 Ohms=0.33 Amps. Accordingly, current measurements of 0.1 Amps and 0.3 Amps from the IT test signals during the treatment would indicate the treatment progress of 8% ((0.1-0.08)/(0.33-0.08)) and 88% ((0.3-0.08)/(0.33-0.08)), respectively.

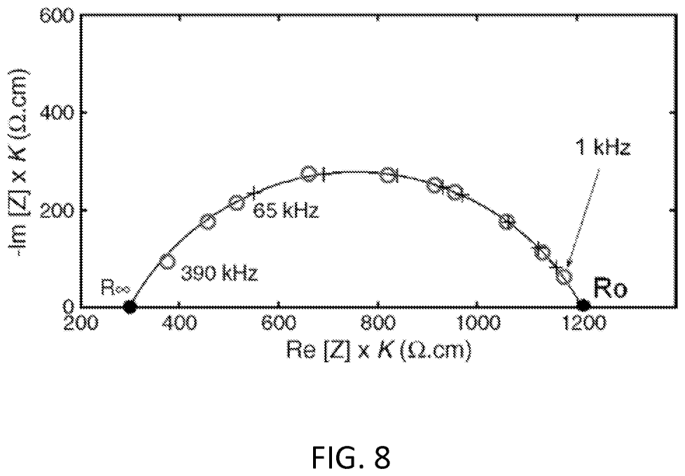

In an alternative to or in addition to applying a test signal at a single frequency, a plurality of test signals at different frequencies can be applied prior to and during the treatment procedure. FIG. 8 illustrates a graph of predicted impedance values across a frequency spectrum as predicted by a Cole model and superimposed impedance values from an actual rat liver prior to electroporation. As the electroporation pulses are applied, the circular shaped curve becomes narrower with real impedance values at lower frequencies approaching those of higher frequencies.

To monitor the progress and determine the progress percentage for display on the display 11 and to determine an end point of the treatment procedure, the reduction of the real part of the impedance values at various test signal frequencies can be monitored. As an example, an AC test signal at frequencies of 1 KHz, 10 KHz, 100 KHz, 1 MHz, 10 MHz, 100 MHz and 300 MHZ (both pre-treatment and intra-treatment) may be used. By experiment and model calculations, the real impedance values at the end of the treatment procedure at those frequencies can be obtained and stored in the memory 44. At step 84, the AC test signals can be applied sequentially in a frequency sweep prior to the treatment and corresponding impedance values (particularly the real part) are obtained and stored. As the electroporation pulses are delivered, the AC test signals at the same frequencies between electroporation pulses are applied and the real impedance values are obtained. They are then compared to the stored values in a similar manner as discussed above to obtain the progress percentage and to determine the end point of the treatment procedure. This method can be more robust than the others as confirmation of the progress level and end point are made at multiple frequencies.

In addition, the imaginary component from the test signals at the various frequencies can be monitored as well, which could serve as a surrogate for changes due to temperature rise. For example, if the imaginary component of the sweep increases by an average of 15% over the different frequencies (or at the peak value from the plot), the target final impedance values from the IT test signals can be adjusted by the 15% as well (i.e., shift down the final impedance value from 150 ohm to 150−150*0.15=127.5 ohm).

Alternatively, rather than applying test signals in step 90, the treatment control module 54 uses the electroporation pulses themselves (as applied in step 88) to measure the real impedance values based on the voltage applied and the current sensed by the sensor 73 as the electroporation pulses are being applied.

In step 94, the treatment control module 54 determines whether the impedance value (real part of the impedance value obtained in step 90) of the IT test signal has reached the baseline impedance value (imaginary part of the complex impedance obtained from step 84) of the PT test signal. Specifically, the module 54 determines whether the impedance value from step 90 is less than or equal to the baseline value from step 84. If the answer is NO, then the method under the control of the treatment control module 54 automatically goes back to step 88 where the electroporation pulses are applied again.

If the answer is YES, however, the treatment control module 54 executes a second comparison step (step 100) to make sure that the treatment has reached an end. In step 100, the module 54 determines whether the change of real impedance values from successive IT test signals is less than a preset threshold value. As discussed earlier, preferably, SMA or EMA values are used to remove signal-to-signal fluctuation. For example, the preset threshold value may be 25 Ohms, which may be preset or user-programmed or user-adjusted. The assumption is that when the real impedance value does not vary by much, e.g., less than 25 Ohms, between successive IT test signals, then the treatment has reached an end.

If the answer is NO, then the method goes back to step 88 where the electroporation pulses are applied again. If the answer is YES, however, the treatment control module 54 determines that the end of treatment has been reached. As a result, in step 102, the treatment control module 54 sets an End-of-Treatment flag.

In step 98, the module 54 terminates the treatment procedure. Alternatively, rather than terminating the procedure, the treatment control module 54 may provide an option to the user/physician to complete the programmed number of electroporation pulses. Of course, if there are multiple pairs of electrodes and treatment procedure for the target region represented by only one pair is completed, the method automatically goes to the next pair of electrodes and repeats the steps starting at step 84 or 88.

As a realistic example, assume that the baseline impedance value (imaginary part of the complex impedance obtained from step 84) is determined to be 150 Ohms. Since the AC signal effectively short circuits the cell membrane, the baseline imaginary impedance value generally represents the electrical resistance of the tissue cells without the effects of their membranes. At the beginning of the treatment, as the electroporation pulses are applied in step 88, the impedance (real part) of the IT test signal may be relatively high, e.g., 650 Ohms, because the cell membranes block the flow of electricity. However, as treatment progresses, the electroporation pulses start to puncture holes in the membranes and the electrically conductive fluid from inside the cells starts to flow out through the punctured holes. This results in a conductance increase and an impedance (real impedance) decrease. At some point during the delivery of treatment pulses, the real impedance reaches the baseline impedance value of 150 Ohm or the scaled targeted value (e.g., 130 Ohms) based on change in temperature factor. At that point, the treatment control module 54 determines that the end of treatment has been reached.

In an alternative embodiment, the comparison in step 94 is sufficient to determine that the end of treatment has been reached and step 100 is not executed. Conversely, in another alternative embodiment, the comparison in step 100 is sufficient to determine that the end of treatment has been reached and step 94 is not executed.

In yet another alternative embodiment, steps 94 and 100 are reversed such that the change of real impedance values from successive IT test signals need to fall below a preset threshold value before the comparison of whether the impedance value (real part of the impedance value obtained in step 90) of the IT test signal is less than or equal to the baseline impedance value (imaginary part of the complex impedance obtained from step 84) of the PT test signal occurs.

In another aspect of the present invention, IRE and other electroporation procedures would benefit greatly by utilizing the available tissue property data to gain insight into the response of the tissue relative to expectations (e.g., tissue response such as current and impedance from PT alternating current test signals and IT test signals) and adjust or control the electroporation protocol accordingly. By involving actual tissue response for the case and electrode pair at-hand, it should be possible to predict completeness and dimensions of ablation with higher reliability and tighter tolerances than that using a prescribed pulsing protocol alone.

In another aspect of the invention, the pulse metrics and their trends are incorporated with the user-input electrode separation distances in a system analyzer to predict lesion dimensions for the present electrode pulsing pair. A detailed discussion of predicting lesion dimensions is disclosed in PCT International Application Number PCT/US10/29243, filed Mar. 30, 2010 and entitled “System and Method for Estimating a Treatment Region for a Medical Treatment Device and for Interactively Planning a Treatment of a Patient”, which is incorporated herein by reference.

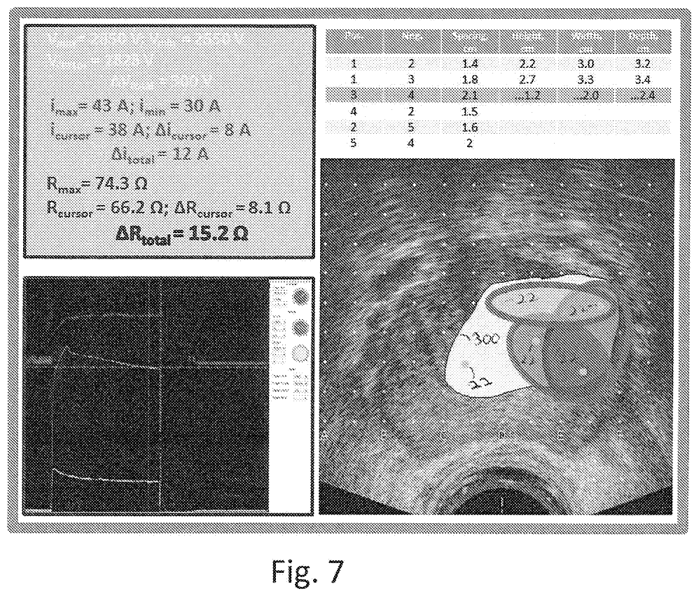

As shown in FIG. 7, as the procedure progresses for the given electrode pair and the pulse metrics reflect growth in the lesion, the treatment control module 54 will update with the most current predictions of ablation dimensions, thus enabling the user to determine when the pair has attained satisfactory ablation dimensions and the electroporation protocol can progress onto the next electrode pair. The dimensions are given as tabulated data and also included on a graphical depiction of electrode locations, both of which are in the user interface screen.

The treatment control module 54 also records the accumulation of ablated areas for each electrode pair in the protocol, enabling the user to monitor their superimposition and ensure the overall ablation protocol addresses all targeted regions. As shown in FIG. 7, the lesion 300 to be ablated is shown with electrode 22 placements and the corresponding predicted ablation area for each pair of electrodes, which are superimposed on the actual imaged tissue area (e.g., ultrasound image) in real-time.

The treatment control module 54 is able to control the progress of the electroporation protocol by directing the therapeutic pulse generator. In essence, once a satisfactory ablation zone has been achieved for a given electrode pair, the control module 54 is able to rapidly move the pulses to the next electrode pair to continue the procedure. This includes detection of when the lesion is too small and the system can control the generator to deliver additional pulses or ones of greater magnitude (higher voltage, longer pulse length).

The treatment control module 54 interprets the pulse metric data in relation to the tissue type, electrode separation from the generator input, and previous pulse data. This information is integrated with previously calibrated information that correlates ablation dimensions with these metrics. The result of the integration is to predict the ablation dimensions for the given electrode pair. This dimension prediction is updated with every pulse as the pulse metrics continue to change through the procedure with the help of IT test signals. This information is sent to a graphical user interface of the treatment control module for display in the monitor 11.

The system 2 has a feedback screen, which is a graphical user interface that conveys all relevant information regarding the pulse analysis and ablation zone predictions. This includes the previous pulse waveforms of voltage, current, and resistance calculation (bottom left portion of FIG. 7). It also retrieves the relevant data from the pulse metrics stored in the memory 44 and conveys this information in a tabulated form for the user to see (top left portion). The ablation zone predictions are conveyed to the user for each electrode pair undergoing pulsing during the electroporation protocol, where the previous final zones are stored, and the active electrode pair has dimensions that will grow as pulsing and electroporated volume continues, as predicted by the pulse metrics (top right portion). Finally, an overlay of a medical image, such as ultrasound, CT, or MRI is displayed, where the user can trace the region of interest, and also displays the electrode array provided from the generator input (bottom right portion). The predicted ablation zones for each electrode pair previously performed (electrode pairs 1-2 and 1-3) are superimposed on this image, as well as the currently active electrode pulsing pair (electrodes 3-4), which will change in dimension based on the ablation zone predictions as the pulse metric data changes. The ablation zone predictions can be calculated based on the test signals (PT and IT signals) as the treatment progresses. For example, the calculations can be made based on adjustments to the Cassini oval equations as described in applicant's own PCT International Application Number PCT/US10/29243, filed Mar. 30, 2010 and entitled “System and Method for Estimating a Treatment Region for a Medical Treatment Device and for Interactively Planning a Treatment of a Patient”, which is incorporated herein by reference.

In addition, the treatment control module 54 is used to guide the progression of the electroporation process based on data provided by the user and the sensor 73 data. The module controls the electroporation pulse generator 10 by altering the inputs to reflect the intentions of the user. This includes changing the pulse parameters to increase the ablation zone 300 if the pulse metrics indicate that the zone is too small as visually seen on the display 11 (see FIG. 7) and greater voltage, pulse length, or pulse number is necessary. In addition, the module 54 determines when the ablation zone for a given electrode pair has reached a satisfactory size for the demands of the user, and indicates to the generator 10 to move to the next electrode pair in the protocol sequence.

The above disclosure is intended to be illustrative and not exhaustive. This description will suggest many modifications, variations, and alternatives may be made by ordinary skill in this art without departing from the scope of the invention. Those familiar with the art may recognize other equivalents to the specific embodiments described herein. Accordingly, the scope of the invention is not limited to the foregoing specification.
1-20. (canceled)

21. A method comprising:
applying, via at least one electrode, a plurality of electrical pulses to tissue of a patent and inducing irreversible electroporation of cells of the tissue or the tissue;
determining an impedance of the tissue associated with the plurality of electrical pulses;
determining whether the measured impedance is within an acceptable threshold range,
wherein, when the impedance is within the acceptable threshold range, continuing to apply the plurality of electrical pulses, and
wherein, when the impedance is outside the acceptable threshold range, stopping application of the plurality of electrical pulses.

22. The method of claim 21, wherein the plurality of electrical pulses have a frequency of 1 KHz, 10 KHz, 100 KHz, 1 MHz, 10 MHz, 100 MHz or 300 MHZ.

23. The method of claim 21, wherein the plurality of electrical pulses have a frequency of at least 1 MHz.

24. The method of claim 21, wherein the at least one monopolar electrode includes a pair of electrodes including a positive electrode and a negative electrode.

25. The method of claim 21, wherein the at least one monopolar electrode includes a probe and a ground pad electrode.

26. The method of claim 25, further comprising:
positioning a ground pad electrode at an exterior of the patient; and
advancing the probe to the tissue of the patient.

27. The method of claim 21, further comprising:
applying one or more pre-treatment test signals and one or more intra-treatment test signals.

28. The method of claim 21, wherein the acceptable threshold range is approximately 150 Ohms.

29. The method of claim 27, further comprising:
determining an effect on the tissue by the plurality of electrical pulses based on a difference between one or more electrical properties of the tissue associated with application of one or more of the pre-treatment test signals to the tissue and one or more electrical properties of the tissue associated with application of one or more of the intra-treatment test signals to the tissue.

30. The method of claim 21, further comprising:
measuring an electrical current associated with the application of the electrical pulses via a current sensor; and
applying an algorithm to evaluate the electrical current.

31. The method of claim 30, wherein measuring a voltage associated with the application of the electrical pulses via a voltage sensor.

32. The method of claim 31, wherein the impedance is determined based on the measured electrical current and measured voltage.

33. A method of treating a tissue of a patient, the method comprising:
positioning a ground pad electrode at an exterior of the patient;
advancing at least one electrode to the tissue to be treated;
activating a generator to apply a plurality of electrical pulses, via the at least one electrode, to the tissue and inducing irreversible electroporation of cells of the tissue or the tissue;
continuing to apply the plurality of electrical pulses when a measured impedance is within an acceptable threshold range; and
stopping application of the plurality of electrical pulses when the measured impedance is outside of the acceptable threshold range,
wherein a controller is operatively coupled to the generator and configured to:
measure the impedance of the tissue associated with the plurality of electrical pulses; and
determine whether the measured impedance is within the acceptable threshold range.

34. The method of claim 33, wherein the plurality of electrical pulses have a frequency of 1 KHz, 10 KHz, 100 KHz, 1 MHz, 10 MHz, 100 MHz or 300 MHZ.

35. The method of claim 33, wherein the plurality of electrical pulses have a frequency of at least 1 MHz.

36. The method of claim 33, wherein the controller further:
generates one or more pre-treatment test signals to the tissue and one or more intra- treatment test signals to the tissue.

37. The method of claim 36, wherein the controller further:
determines an effect on the tissue by the plurality of electrical pulses based on a difference between one or more electrical properties of the tissue associated with application of one or more of the pre-treatment test signals to the tissue and one or more electrical properties of the tissue associated with application of one or more of the intra-treatment test signals to the tissue.

38. The method of claim 33, wherein the controller further:
measures an electrical current associated with the application of the electrical pulses via a current sensor; and
applies an algorithm to evaluate the electrical current.

39. The method of claim 38, wherein the controller further. measures a voltage associated with the application of the electrical pulses via a voltage sensor.

40. The method of claim 39, wherein the impedance is determined based on the measured electrical current and measured voltage.
